(19) United States
(12) Patent Application Publication
(10) Pub. No.: US 2026/0356571 A1
(43) Pub. Date: Nov. 12, 2026

(54) DISPENSING SYSTEMS AND METHODS FOR COMPRESSIBLE ARTICLES

(71) Applicant: 3M INNOVATIVE PROPERTIES COMPANY, St. Paul, MN (US)

(72) Inventors: Marco Decca, Indianapolis, IN (US); Jeffrey L. Hamer, Springville, IN (US); Mark A. Yoder, Zionsville, IN (US); Jacob H. Ely, Carmel, IN (US)

(21) Appl. No.: 19/137,248

(22) PCT Filed: Dec. 18, 2023
(86) PCT No.: PCT/IB2023/062885

§ 371 Date: Jun. 9, 2025

Related U.S. Application Data
(60) Provisional application No. 63/387,979, filed on Dec. 19, 2022.

(51) Int. Cl. B65D 83/08; B65D 25/54

(52) U.S. Cl. CPC B65D 83/0841; B65D 25/54; B65D 83/0894

(57) ABSTRACT

A hearing protection dispensing device is presented that includes a housing (500) with a mounting feature (520) and a stationary separation feature (510). The dispensing device is configured to receive an assembly of packaged hearing protection devices. The assembly of packaged hearing protection devices includes a first package coupled to and stacked on top of a second package, the second package being coupled to and stacked on top of a third package. The stationary separation feature (510) interacts with a coupling between packages in the assembly, such that the stationary separation feature releases a single package at a time.

### BACKGROUND

Many personal protective equipment devices are single-use articles. Users get these devices from dispensers located within a workplace. Often, such devices are distributed in large volumes to a substantial number of users. Dispensers may be used for such purposes. A dispenser may dispense more than 100 articles in a day.

### SUMMARY

A hearing protection dispensing device is presented that includes a housing with a mounting feature and a stationary separation feature. The dispensing device is configured to receive an assembly of packaged hearing protection devices. The assembly of packaged hearing protection devices includes a first package coupled to and stacked on top of a second package, the second package being coupled to and stacked on top of a third package. The stationary separation feature interacts with a coupling between packages in the assembly, such that the stationary separation feature releases a single package at a time.

Dispensing systems and methods described herein provide a user friendly way to obtain single-use articles while reducing the spread of germs. Articles dispensed using systems and methods herein are single touch, such that a user only touches a package containing the articles they will use, and does not need to touch the dispenser, or any other articles. Systems and methods herein also provide compressible articles without causing damage to the article being dispensed. Additionally, dispensers herein are low maintenance, not requiring a power source or complicated dispensing mechanisms that can break down.

### BRIEF DESCRIPTION OF THE DRAWINGS

FIGS. 1A-1D illustrate single-use hearing protection devices that may be dispensed using dispensing systems and methods herein.

FIG. 2 illustrates a method of dispensing a single-use article in accordance with embodiments herein.

FIG. 3 illustrates a schematic of compressible articles as would be arranged within dispensing systems and methods of embodiments herein.

FIG. 4 illustrates a method of restocking articles in a dispenser in accordance with embodiments herein.

FIGS. 5A-5B illustrate dispensing systems in accordance with embodiments herein.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

Safety protection devices, such as earplugs, are routinely used in consumer, commercial, and industrial environments. Often, earplugs are distributed in large volumes to a substantial number of users. For example, in a factory or manufacturing setting, earplugs may be provided to all workers and visitors in compliance with safety regulations. However, while described herein is the example of earplugs, it is expressly contemplated that other single-use consumables may also be similarly dispensed in large volumes.

Common modes of earplug distribution include box distribution and mechanical dispenser distribution. One form of box distribution consists simply of a box containing numerous earplugs placed in an open condition in an accessible area. This would include, for example, a box of one-hundred earplugs (individual earplugs or corded pairs, packaged or unpackaged) placed on a table in a work room with the box top open for access by workers.

However, safety requirements have increased in response to COVID variants. A centrally located box distribution requires workers to reach in and potentially touch multiple earplug packages before pulling out the pair they will use. Similarly, a vending machine or other mechanical dispenser may also result in multiple users touching the same buttons, levers or other actuation devices, which is not desired. Similarly, however, it is desired that a dispenser be mechanically simple and not require power or have components that can break down, as potentially hundreds of articles need to be dispensed daily. However, the dispenser also needs to be easily installed, stocks and restocked as needed.

It is also desired to regulate the number of earplugs distributed per user, such that a dispenser dispenses a single pair of earplugs each time a user engages the dispenser. Further, many earplug models have a cord extending between a pair of earplugs. It is important that cords not get tangled between pairs of earplugs.

In the case of box distribution of packaged earplugs, the box arrangements do not make provision for collection, disposal, or recycling of earplug packaging. Thus, when a packaged earplug pair is accessed from the box, the earplugs are removed from the package and then often the package is not properly disposed or recycled (e.g., the package is left on a table, dropped on a floor, or placed in a rubbish bin instead a plastic recycling receptacle). The result is an untidy dispensing area littered with spent packaging which is not properly collected for recycling.

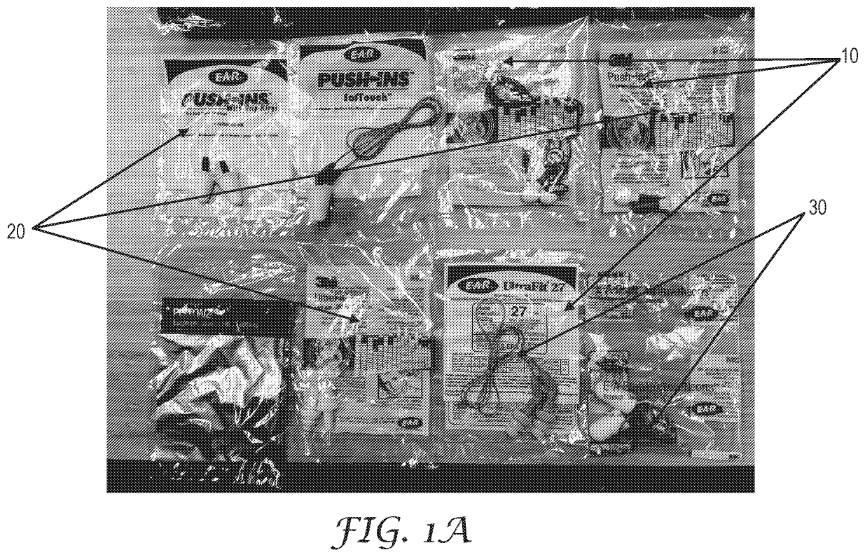

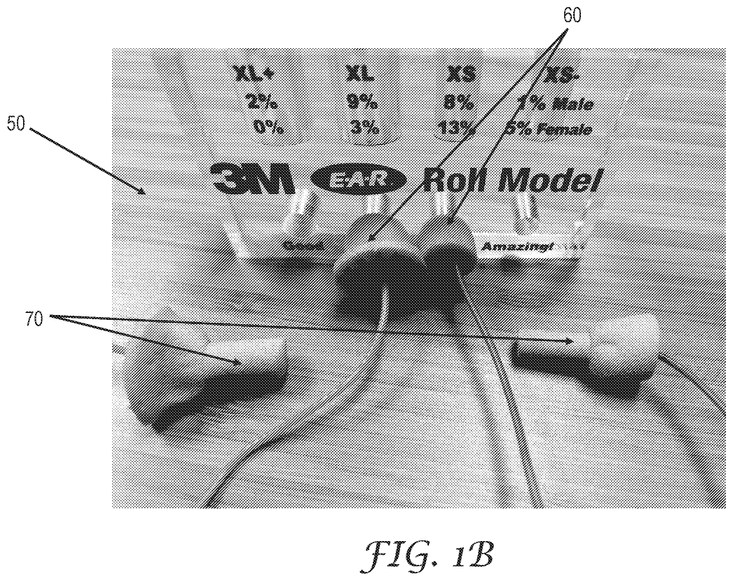

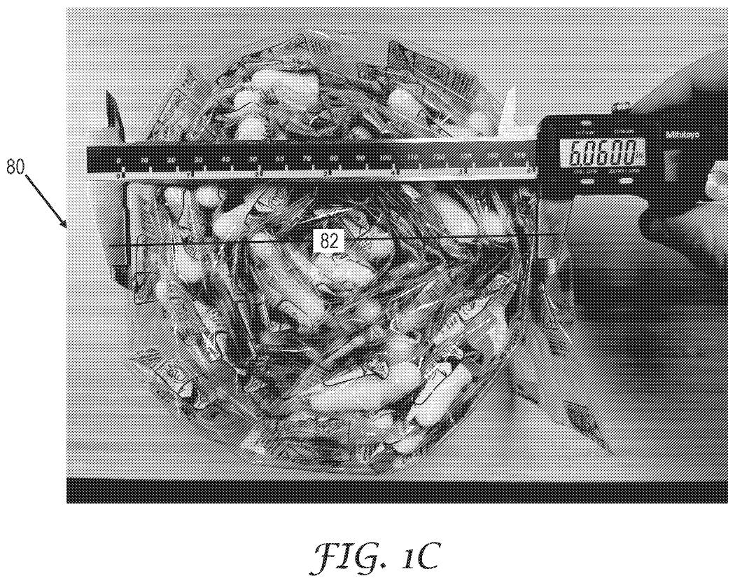

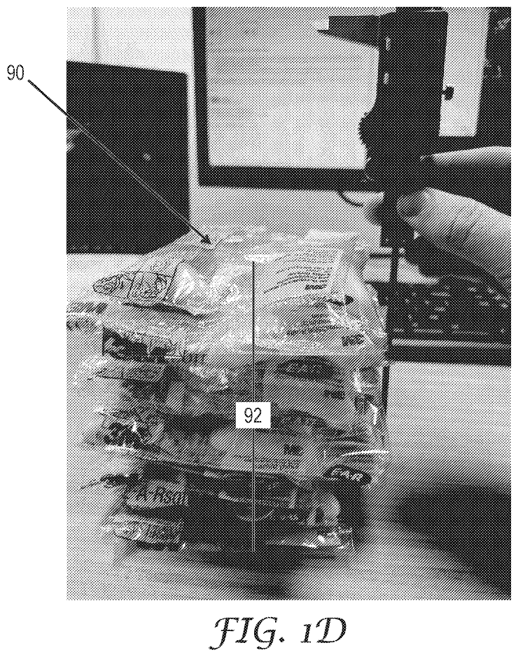

FIGS. 1A-1D illustrate single-use hearing protection devices that may be dispensed using dispensing systems and methods herein. FIG. 1A illustrates eight different hearing protection devices 10, each with different features, all stored within a packaging 20. Packaging 20 may be plastic or another suitable material, and may be clear, as illustrated, opaque or colored. Some of the devices 10, as illustrated, have cords 30. It is noted, when examining devices 10, that the in-ear portion of each of devices 10 take up a small portion of an area of each package 20. This causes earplugs 10 to potentially occupied different spaces in adjacent packages, which can result in a difficult time stacking or otherwise packaging devices 10. Some of devices 10 are made from compressible foam, others are formed from a flexible polymer. Some are formed of multiple materials, such as a stem that varies from a plug.

FIG. 1B illustrates a particular problem facing compressible materials. Devices 70 were inserted into a Roll Model 60 for an extended period of time before being removed. Image 50 illustrates devices 70 an hour after being removed from device 60. A foam that has been continuously compressed may not return to its expanded shape. While foam earbuds are meant to be compressed for use (e.g. rolled down), they are not intended to be compressed for an extended period of time. It is for this reason that such devices are single-use. After an entire shift in a user's ear, they may, as devices 70, experience some permanent deformation.

Similar concerns exist for noncompressible, but otherwise malleable or deformable devices. For example, it is not desired for a polymer earplug to maintain a crushed position while in transit, as it may permanently deform the plug.

FIG. 1C illustrates difficulties presented in packaging deformable or breakable articles. In order to allow for easier dispensing, it may be the case that packaged articles be coupled together, such that dispensing of one causes a second to become available. A roll of packaged articles cannot be wound overly tight, as it could cause unwanted compression or deformation of the articles. However, as illustrated, roll 80 has a diameter 82 that increases with each winding. FIG. 1C illustrates a roll of 50 earplugs, which has a diameter 82 of 6.06 inches. A package containing a roll of articles increases in two dimensions—length and width, while maintaining a constant thickness. This can result in either too small a number of articles to be useful—requiring constant replacement, or a package that is too large to easily mount or replace.

FIG. 1D illustrates the same number of packaged articles, 50, as FIG. 1C. However, stack 90 has a height 92 of 4.556 inches. As each packaged article is stacked on top of the next, the only dimension that changes is height 92, as the length and width of stack 90 is dictated by the size of the packaging. Additionally, gravity may help to provide some compressive force to cause earplugs to move within packaging such that articles in adjacent packages are not directly on top of one another, and height 92 is reduced. As illustrated in the comparison between FIGS. 1C and 1D, a stack of articles is preferred to a roll for its compactness.

While the example of earplugs is discussed in depth herein, other safety protection devices, beyond earplugs, include, for example, safety eyewear, respirators, ventilators, safety gloves, and various components and accessories of thereof may also benefit from dispensing systems and methods herein. These safety protection devices are typically distributed on site by an open-box type arrangement as discussed above with respect to earplugs. For example, at a work site, a box containing a plurality of safety eyewear devices is simply placed at an easily accessible area, such as on a table, and the box top is placed into an open position to expose the contents therein. Users simply reach into the box to access the safety eyewear devices. This open-box distribution presents the same disadvantages previously described concerning the box distribution of earplugs. That is, sanitary conditions may not be maintained, there is no control of the quantity of items distributed, associated packaging is not collected for recycling, etc.

Accordingly, there is a need for dispensing system which can store fragile, deformable or compressible safety protection devices of any form, shape, and size (e.g., earplugs that are corded, stemmed, etc., safety eyewear, etc.), which can readily and easily dispense such devices in an unpackaged condition upon demand by a user, and which dispenser mechanism includes waste and recycling provisions for the empty packaging, where the safety protection devices are maintained in a sanitary condition prior to dispensement and where the quantity of dispensed devices is controlled.

However, it is noted that a stack is less stable than a roll of articles, and may collapse or be knocked over. A dispensing system and method is needed that provides a single article at a time. Such systems and methods are described herein.

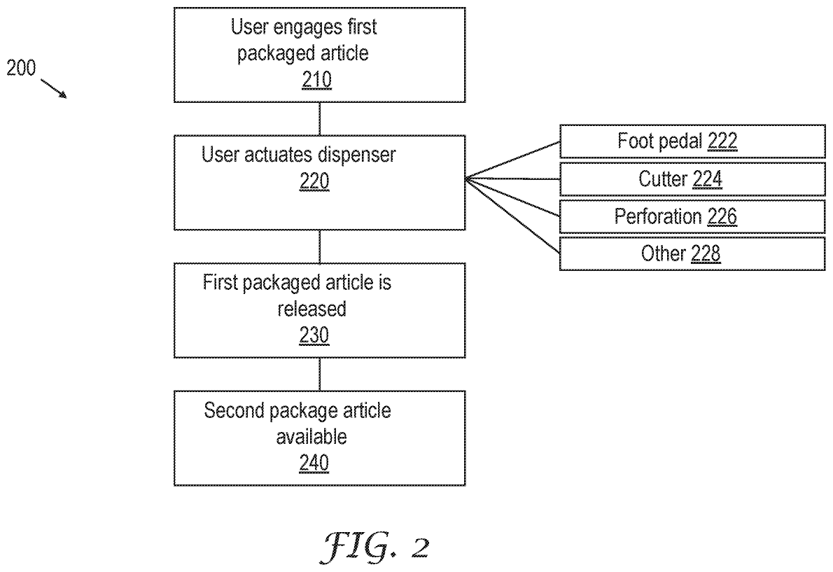

FIG. 2 illustrates a method of dispensing a single-use article in accordance with embodiments herein. As described herein, a method 200 is provided for hygienically dispensing a single packaged article at a time.

In block 210, a user engages a first packaged article. As described herein, the first packaged article may extend from a dispensing unit such that a user can take hold of it.

In block 220, a user actuates a dispenser. Actuating may include the user engaging a mechanical device that separates the first article from a successive article. For example, a cutting element 224 may be available such that as a user pulls the article at a certain angle and force, it tears the packaging, similar to a serrated blade present in an aluminum foil, tape, or wrap dispenser. The cutting element 224 may also be mechanically moved, for example by the packaged articles aligning over a gear system that causes a spring to wind and, when a packaged article has moved into position, release and provide a serrated blade across the packaging to cut the first article free from a second article. A mechanical actuator separate from the dispenser, such as a foot pedal 222, may also be used. In some embodiments, a perforation 226 is present between adjacent packaged articles that allows for more easy separation. The perforation 226 may be present in conjunction with another mechanical separation device 228 or cutter 224.

In block 230, the first packaged article is released from the dispenser. Releasing may include perforations tearing, a packaging being cut or another suitable mechanism that separates a first packaged article from a subsequent packaged article.

In block 240, a second packaged article is available for a user to engage. In some embodiments, blocks 230 and 240 occur simultaneously, such that as the first package is being cut free, torn away, or otherwise released, the second becomes available for user engagement. Additionally.

It is also expressly contemplated that blocks 230 and 240 may happen in response to an action taken in block 220. For example, in one system illustrated below, a user pulling on a packaged article causes a mechanical separation device to tear perforations and, as the first packaged article comes free, the second moves into position for user engagement.

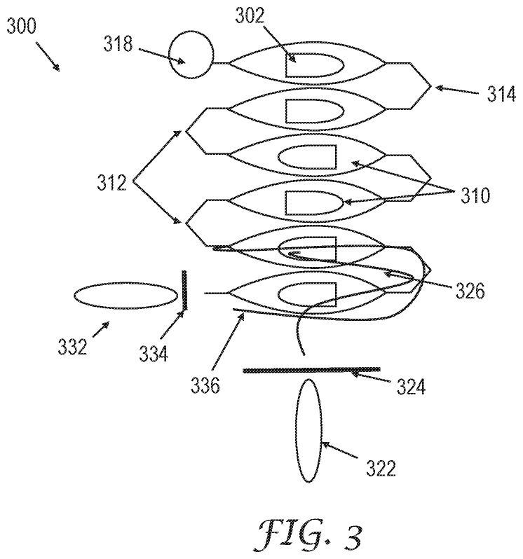

FIG. 3 illustrates a schematic of compressible articles as would be arranged within dispensing systems and methods of embodiments herein. Arrangement 300 may be provided in a container for shipping, or may be presented to a user in a dispenser. A number of articles 302 are housed within packaging 310. Packages 310 are coupled together by coupling 314. As illustrated, packaging is stacked such that couplings 314 are on alternating sides-e.g. the particles are stacked in a “zigzag” pattern. Couplings 314 may include sealed portions of packaging material 310, or another coupling mechanism between adjacent packages 310. A separation feature 312 may be present, in some embodiments, such as a perforation or aperture. A separation feature 312 may also be a colored indicator of where a user should apply a cutting mechanism.

In the arrangement 300, there are two potential ways an article may be separated-article 322 follows path 326, and exiting through the bottom of a dispenser, while article 332 follows path 336, exiting a side of a dispenser.

As illustrated by path 326, an orientation of a packaged article 322 changes by about 90° along path 326. Article 322 passes a separation mechanism 324, which may be a stationary separator, such as a serrated blade or a shaped separator that causes perforations to tear, or may be a separation mechanism that releases a cutter in response to an actuation mechanism.

As illustrated by path 336, an orientation of a packaged article 332 may change 180° while in the dispenser. In some cases, article 332 may rotate another 90° (or back 90°) and hang down (e.g. parallel to article 322) as a result of gravity. A separation mechanism 334 may be built into a dispenser. Separation mechanism 334 may be a stationary separator, such as a serrated blade or a shaped separator that causes perforations to tear, or may be a separation mechanism that releases a cutter in response to an actuation mechanism.

One problem facing dispensers is the restocking process. An advantage of embodiments herein is the ability to increase a number of packaged articles in a single dispenser by increasing only one dimension-the height-of said dispenser. However, restocking still presents an issue as, for a last package 310 of assembly 300, will not automatically pull out a first package of a restock. It is desired for restocking to be easy-ideally such that a new box of packaged articles can be opened and fed directly into a dispenser such that arrangement 300 is maintained.

Therefore, in some embodiments, a restock connector 318 is present at the end of a set of packaged articles. The restock connector 318 should be a connection mechanism that can attach to a corresponding connector on a first packaged article of a next set of packaged articles. The connection should be mechanically simple and quick to implement for a user. Connector 318 may be a mechanical fastener, such as hook-and-loop, a snap, a zipper, or another suitable mechanism. Connector 318 may also be an adhesive based connector-e.g. with an adhesive strip on either or both of a last package of a first assembly 300 and a first package of a second assembly 300. Other connection mechanisms, such as magnets or other suitable options may also be used.

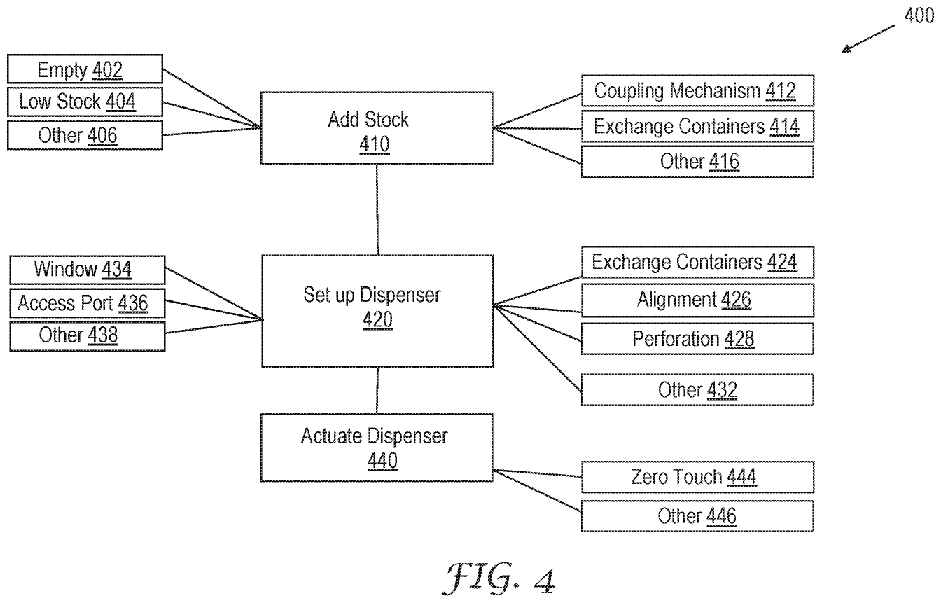

FIG. 4 illustrates a method of restocking articles in a dispenser in accordance with embodiments herein. As noted above, it is important that systems and methods herein allow for easy, quick restocking. Method 400 describes a restocking operation that happens quickly with low effort for a user.

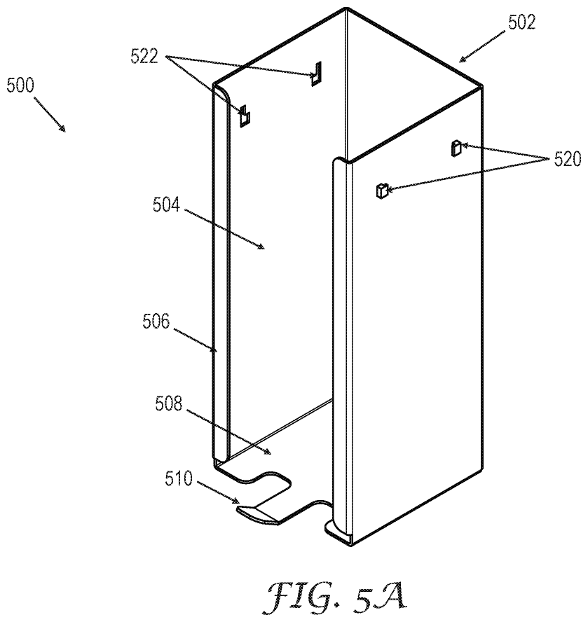

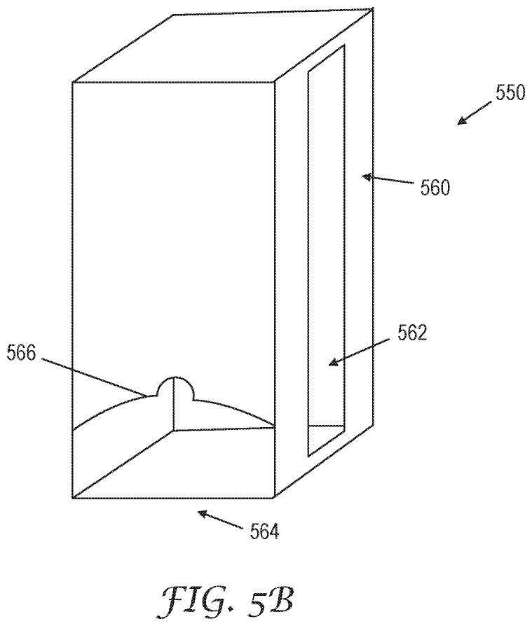

In block 410, stock is added to a dispenser. Stock may include, for example, an assembly of packaged articles that are coupled together in a zigzag orientation. It is important that the orientation of the packaged articles be maintained. Therefore, it may be desired to couple the new assembly to the last article of the previous assembly, using a coupling mechanism 412. Coupling mechanism 412 may include adhesive, a mechanical coupling such as a snap or hook and loop, or another suitable coupling mechanism. It is also envisioned that, in some embodiments, a container of articles can be exchanged, as indicated in block 414, when the current container is empty. As described below with respect to FIGS. 5 and 6, a package containing the assembly of articles may be placed within a mounted dispenser. Stock may be added in other ways, as indicated in block 416.

It is expressly contemplated that a dispenser can be restocked when it is empty 402 as well as when stock is low 404. When stock is low, it may be necessary to couple the end of a current assembly to a new assembly in order for the first packaged article of the new assembly to be available for dispensing. However, it is expressly contemplated that stock may be added at a different triggering event 406.

In block 420, the dispenser is setup for dispensing. For example, it may be necessary to exchange a used container with a new container, as illustrated in block 424. For example, as illustrated in FIGS. 5-6 below, the dispenser may ship with the packaged articles (e.g. as a container) and may be converted into a dispensing unit that is inserted into a mounting unit, or directly mounted to a wall. In such embodiments, it may be possible to create an inventory window 434, an access port 436, or another feature 438. For example, the container may come with perforations 428 allowing for a user to easily punch or tear out a window 434 that allows a user to see when inventory runs low. Similarly, a user may punch or tear out an access port 436 that allows access to the packaged articles. The access port 436 may include a separation mechanism, e.g. a serrated blade or a mechanical separation mechanism, such as a protrusion that engages with an aperture in the packaging. However, it is expressly contemplated that the separation mechanism may be part of a mounted dispenser, and not part of a container, such that only the access port 436 needs to be formed.

Setting up a dispenser may also include an alignment step 426. For example, if a mechanical separation mechanism is present, it may be necessary to align the first packaged article such that the separation mechanism is in position. For example, if a cutting element is spring-loaded, it may be necessary to feed in the packaged articles such that the spring-loaded mechanism will actuate properly. For a stationary separation mechanism, it may be necessary to place the first packaged item correctly.

Other actions 432 may be needed to set up a dispenser for operation.

However, it is expressly contemplated that no setup is required, in some embodiments, and stock is added in block 410 and the method proceeds directly to actuation in block 440.

In block 440, the dispenser is actuated. As described herein, the dispenser itself, once set up, is a zero touch dispenser 444. Zero touch refers to the number of touches the user has with the dispenser itself. With respect to the dispensed article, the user touches only the packaging for the article that is dispensed to them, and no other packaging. This reduces potential spread of germs between users. Actuating may include other features 446.

It is noted that setup of the dispenser may result in additional touches applied to a first packaged article, e.g. during connection of coupling mechanism 412. It may be desired to limit restocking procedures to an industrial hygienist, or to require that a user doing a restocking operation wear gloves, wash their hands, or apply antibacterial or disinfecting material to their hands prior to block 410.

FIGS. 5A-5C illustrate dispensing systems in accordance with embodiments herein. FIG. 5A illustrates a dispenser housing 500. Housing 500 may receive packaged articles directly, as illustrated in FIG. 5C, or may receive a container of packaged articles, as illustrated in FIG. 5B and FIG. 6. Guards 506 may be present around an opening 504 such that a container, or assembly of packaged articles, can be easily inserted into housing 500.

Dispenser housing 500 may have mounting features 520 that allow for additional dispensers 500 to be mounted side by side. Such an arrangement may reduce how often restocking needs to occur, and allows for restocking of multiple dispensing units 500 to be done quickly. Mounting features 520 may be received by receiving apertures 522. In FIG. 5A, a pair of features 520 and apertures 522 are illustrated. However, it is expressly contemplated that any number of mating features 520, 522 may be present. For example, a single mating feature 520 may be sufficient, such as a bar that is received by an aperture 522. More mating features 520 may also be used, such as three or more apertures 520. Dispenser housing 500 may also have mounting features (not shown) on side 502, opposite opening 504, such that dispenser housing 500 can be securely mounted to a wall.

Dispenser housing 500 may also have a separation mechanism 510. In the embodiment illustrated in FIGS. 5A and 5C, separation mechanism 510 is an angled protrusion that is received by an aperture in packaging of packaged articles (as illustrated and described in FIG. 5C) and helps in the separation of a first packaged article from a second packaged article. However, while protrusion 510 is angled upward from a bottom plane 508 of dispenser 500 in FIG. 5A, in some embodiments it is substantially parallel to plane 508, or angled down from plane 508.

FIG. 5B illustrates a schematic of another dispenser housing 550. Instead of guards, dispenser 550 has a solid front face 560 with an inventory window 562 that allows a user to see how many packaged articles remain. FIG. 5B does not show an access port, but it may be located on face 560 or on a bottom surface 564. A separation mechanism may also be present, though not shown in FIG. 5B.

FIG. 5C illustrates a prototype of a dispenser 570. Dispenser 570 illustrates an embodiment where packaged articles are inserted directly into dispenser 570, and held in the dispenser by guards 578. A first package 572 engages a separation mechanism 576 using an aperture present in the packaging assembly. The aperture may be a slit or a perforation. As a user pulls on first package 572, separation mechanism 576 keeps packaged article 574 substantially stationary. Packaged article 574 may also be moved forward into position by action of a separation mechanism 576, or by the force of gravity and the weight of packaged articles in dispenser 570. As illustrated in FIG. 5C, dispenser unit 570 may be mounted on a surface, like a table, or to a wall, as discussed previously.

FIG. 6 illustrates a package for compressible articles in accordance with embodiments herein. FIG. 600 illustrates two containers 610, 620 that may be used to ship packaged articles 602. For ease of understanding, packages 602 are illustrated without any articles therein. However, it is expressly contemplated that packages 602 may have articles therein and, therefore, may not be as smoothly packaged as illustrated in FIG. 6.

Containers 610 and 620 each contain an assembly of packaged articles 602. packaged articles are coupled together by couplings 604 that, as illustrated, form a zigzag pattern within containers 610, 620.

Containers 610 and 620, in some embodiments, are resilient containers that have sufficient structure to avoid being crushed and, therefore, crushing packaged articles 602. Containers 610, 620 may serve as a dispenser, for example being formed of plastic and having mounting features (or being shipped with a hook and loop or other attachment mechanism), or being intended to use without mounting—e.g. as illustrated in FIG. 5C.

As described herein, for example with respect to FIG. 5A, containers 610, 620 may be sized to fit within a dispenser housing 500, such that guards 506 hold container 610/620 in place. Housing 500 may be sized to receive both containers 610, 620 in the illustrated configuration.

Containers 610, 620 may be convertible into a dispensing unit, in some embodiments, with an access point 624 or 626 that can be formed by removing a perforated portion of the container such that a first packaged article can be retrieved. Containers 610, 620 may also have a window 622 that can be either built into the container or created during dispenser set up, e.g. by removing a perforated portion of the box (e.g. like a tissue box).

FIG. 6 illustrates two containers 610, 620 as part of a restocking operation. Restocking may occur when container 620 is low, for example. Or, as illustrated, a dispenser may house two containers, such that, when the top container (610) is empty, it can be exchanged and the packaged article assemblies linked together. Other options are expressly contemplated. For example, container 610 is also illustrated as having an optional access port 614 that may be created by tearing along the illustrated perforations. Container 610 may also have an optional window 612.

A first packaged article of box 610, and a last packaged article of box 620, may have a connection feature 618 that allows for the packaged article assembly of box 610 to connect to the packaged article assembly of box 620. This may ensure that the last packaged article of box 620, when removed, causes the first packaged article of box 610 to be properly aligned for dispensing. Connection feature 618 may be a self-assembling connection feature, such as a pair of magnets. Connection feature 618 may include adhesive strips or dots that a user makes available (e.g. by removing a protective liner) and then connects between the two packaged articles. Connection feature 618 may also include a mechanical mechanism-such as a snap, hook and loop, or another suitable mechanism.

It may also be desired to mark packaged articles 602 such that a low inventory is indicated. For embodiments where containers 610, 620 are both shipping containers and dispensers, this may allow for an inventory window to be omitted, potentially improving structural integrity. For example, a last 5, or last 10, or last 10% of packaged articles may have an indicator, such as coloring, words, shapes or other markings, notifying a user that the end of an assembly of packages 602 is approaching. In an embodiment such as that shown in FIG. 6, this may cause a user to remove an empty upper container 610, add a new container 610 fill of packaged articles 602, and actuate coupling mechanisms 618. However, it is expressly contemplated that other restocking options are possible. For example, in an embodiment where container 620 is empty, it may be removed and container 610 inserted into a housing instead.

It is desired that the zigzag pattern formed by packages and couplings 604 be maintained during restocking so that the packaged articles do not get tangled, compressed or otherwise stuck within containers.

A hearing protection dispensing device is presented that includes a housing with a mounting feature and a stationary separation feature. The dispensing device is configured to receive an assembly of packaged hearing protection devices. The assembly of packaged hearing protection devices includes a first package coupled to and stacked on top of a second package, the second package being coupled to and stacked on top of a third package. The stationary separation feature interacts with a coupling between packages in the assembly, such that the stationary separation feature releases a single package at a time.

The hearing protection dispenser may be implemented such that the housing includes a window such that a number of remaining packaged hearing protection devices can be seen.

The hearing protection dispenser may be implemented such that the first package, second package, and third packages include compressible in-ear hearing protection devices.

The hearing protection dispenser may be implemented such that the first, second and third packages include foam ear plugs.

The hearing protection dispenser may be implemented such that each of the packaged hearing protection devices include a pair of in-ear plugs connected by a cord.

The dispenser may be implemented such that the stationary separation feature includes a serrated blade.

The dispenser of any of claims 1-6. The stationary separation feature includes a protrusion that extends from the housing, and wherein the protrusion interacts with an aperture present between adjacent packaged articles.

The dispenser may be implemented such that the protrusion is angled with respect to the housing.

The dispenser may be implemented such that the coupling includes a seal between packages in the assembly, and perforations in the seal.

The dispenser may be implemented such that the assembly of packaged articles forms a zigzag shape in the housing.

The dispenser may be implemented such that the housing includes a guide that holds the assembly of packaged hearing protection devices in position within the housing.

The dispenser may be implemented such that the mounting feature is located on a face of the housing opposite the separation feature and the mounting feature is configured to mount the dispenser to a wall.

The dispenser may be implemented such that the mounting feature is configured to receive a second dispenser such that the dispenser and the second dispenser are mounted side-by-side.

The dispenser may be implemented such that the mounting feature is configured to receive a trash receptacle.

The dispenser may be implemented such that the dispenser housing is free of gears.

The dispenser may be implemented such that the dispenser operates without a power source.

An assembly of packaged hearing protection devices for a dispenser that includes a plurality of hearing protection devices in a packaging sleeve. The sleeve includes a separation between adjacent hearing protection device. The separation includes a plurality of perforations and an aperture, and the aperture is at least twice as long as the average perforation length.

The assembly may be implemented such that the sleeve includes 50 hearing protection devices.

The assembly may be implemented such that the sleeve includes 100 hearing protection devices.

The assembly may be implemented such that the sleeve includes 150 hearing protection devices.

The assembly may be implemented such that the sleeve includes plastic.

The assembly may be implemented such that the separation is a heat seal.

The assembly may be implemented such that the sleeve includes a reusable portion with a resealable closure.

The assembly may be implemented such that the perforations are mechanically formed in the heat seal.

A container for dispensing a hearing protection device is presented that includes a rectangular prism-shaped housing including a height, a length and a width. The length and width are sized to receive a package containing the hearing protection device, such that the length is similar to a package length and the width is similar to a package width. The container is convertible into a dispenser by forming an access port to access the package.

The container may be implemented such that the access port is formed by removing a perforated portion of the container.

The container may be implemented such that the rectangular prism has two end faces with a first area and four side faces with a second area. The perforated portion includes a portion of one of the two end faces.

The container may be implemented such that the rectangular prism has two end faces with a first area and four side faces with a second area. The perforated portion includes a portion of one of the four side faces.

The container may be implemented such that the container includes plastic.

The container may be implemented such that the container includes a paper-based material.

The container may be implemented such that the container includes cardboard.

The container may be implemented such that the length is within 5% of the package length.

The container may be implemented such that the length is within 10% of the package length.

The container may be implemented such that the width is within 5% of the package length.

The container may be implemented such that the width is within 10% of the package length.

A dispensing system for delicate articles is presented that includes a power free dispenser housing. The housing includes: a mounting mechanism, a separation mechanism, and an alignment device that maintains alignment of an assembly of packaged articles. The assembly of packaged articles includes: a packaging sleeve containing a plurality of packaged articles and a plurality of seals separating each of the plurality of packaged articles from an adjacent packaged article. The separation mechanism is configured to interact with each of the plurality of seals to separate a first packaged article from a second packaged article. Prior to interaction with the separation mechanism, the second packaged article is coupled to the first packaged article.

The system may be implemented such that the delicate article is a personal safety article.

The system may be implemented such that the delicate article includes earplugs.

The system may be implemented such that the delicate article includes safety glasses.

The system may be implemented such that the earplugs include compressible material.

The system may be implemented such that the housing has a first surface, a second surface, a third surface and a fourth surface. The first surface is perpendicular to each of the second, third and fourth faces. The second face is opposite the fourth face. The third face is coupled to the second face, on a first edge, and the fourth face, on a second edge.

The system may be implemented such that the alignment device includes a guide protruding from the second face, such that the guide is opposite the third face.

The system may be implemented such that the alignment device includes a second guide protruding from the fourth face, such that the second guide is opposite the first face.

The system may be implemented such that the alignment device is a fifth face, opposite the third face, the fifth face coupled to both the second face, on a fifth face first edge, and the fourth face, on a fifth face second edge.

The system may be implemented such that the fifth face includes an aperture extending from an outer surface to an inner surface, and wherein the aperture has a length and a width.

The system may be implemented such that the housing is formed by removing a portion of container holding the assembly of packaged articles.

The system may be implemented such that the portion is removed such that the assembly of packaged articles can be aligned with the separation device.

The system may be implemented such that the housing includes plastic, a paper-based product or glass.

The system may be implemented such that the housing is transparent.

The system may be implemented such that the housing is opaque.

The system may be implemented such that the system includes an inventory indicator.

The system may be implemented such that the inventory indicator is a viewing window in the housing.

The system may be implemented such that the inventory indicator includes an indication on portion of the packaging sleeve.

The system may be implemented such that the inventory indicator includes color.

The system may be implemented such that the inventory indicator includes letters or symbols.

The system may be implemented such that the separation mechanism includes a protrusion extending from a face of the housing.

The system may be implemented such that the protrusion is angled with respect to the face.

The system may be implemented such that the separation mechanism includes a blade.

The system may be implemented such that the blade is coupled to an edge of the housing.

The system may be implemented such that the separation mechanism is a moving mechanism, and wherein movement is actuated by a user pulling on a first packaged article.

The system may be implemented such that the seal includes an aperture that receives the separation mechanism.

We claim:

1. A hearing protection dispensing device comprising:
a housing with a mounting feature;
a stationary separation feature, wherein the stationary separation feature comprises a protrusion that extends from the housing, and wherein the protrusion interacts with an aperture present between adjacent packaged articles;
wherein the dispensing device is configured to receive an assembly of packaged hearing protection devices, wherein the assembly of packaged hearing protection devices comprises a first package coupled to and stacked on top of a second package, the second package being coupled to and stacked on top of a third package; and
wherein the stationary separation feature interacts with a coupling between packages in the assembly, such that the stationary separation feature releases a single package at a time.

2. The hearing protection dispenser of claim 1, wherein the housing comprises a window such that a number of remaining packaged hearing protection devices can be seen.

3. The hearing protection dispenser of claim 1, wherein the first package, second package, and third packages comprise compressible in-ear hearing protection devices.

4-6. (canceled)

7. The dispenser of claim 1, wherein the protrusion is angled with respect to the housing.

8. The dispenser of claim 1, wherein the coupling comprises a seal between packages in the assembly, and perforations in the seal.

9. The dispenser of claim 1, wherein the assembly of packaged articles forms a zigzag shape in the housing.

10. The dispenser of claim 1, wherein the housing comprises a guide that holds the assembly of packaged hearing protection devices in position within the housing.

11-16. (canceled)

17. A system comprising:
the dispenser of claim 1, and
a container configured to recieve the assembly of packaged hearing protection devices, the container comprising:
a rectangular prism-shaped housing comprising a height, a length and a width;
wherein the length and width are sized to receive a package containing the hearing protection device, such that the length is similar to a package length and the width is similar to a package width; and
wherein the container is convertible into a dispenser by forming an access port to access the package.

18. The system of claim 17, wherein the access port is formed by removing a perforated portion of the container.

19-21. (canceled)

22. A dispensing system for delicate articles, the system comprising:
a power free dispenser housing, wherein the housing comprises:
a mounting mechanism;
a separation mechanism, wherein the separation mechanism comprises a protrusion extending from a face of the housing; and
an alignment device that maintains alignment of an assembly of packaged articles; and
the assembly of packaged articles comprising:
a packaging sleeve containing a plurality of packaged articles;
a plurality of seals separating each of the plurality of packaged articles from an adjacent packaged article; and
wherein the separation mechanism is configured to interact with each of the plurality of seals to separate a first packaged article from a second packaged article, wherein, prior to interaction with the separation mechanism, the second packaged article is coupled to the first packaged article.

23-25. (canceled)

26. The system of claim 22, wherein the housing has a first surface, a second surface, a third surface and a fourth surface, wherein the first surface is perpendicular to each of the second, third and fourth faces, and wherein the second face is opposite the fourth face, and wherein the third face is coupled to the second face, on a first edge, and the fourth face, on a second edge.

27. The system of claim 26, wherein alignment device comprises a guide protruding from the second face, such that the guide is opposite the third face.

28. The system of claim 26, wherein the alignment device is a fifth face, opposite the third face, the fifth face coupled to both the second face, on a fifth face first edge, and the fourth face, on a fifth face second edge.

29. The system of claim 28, wherein the fifth face comprises an aperture extending from an outer surface to an inner surface, and wherein the aperture has a length and a width.

30. The system of claim 22, wherein the housing is formed by removing a portion of container holding the assembly of packaged articles.

31. The system of claim 30, wherein the portion is removed such that the assembly of packaged articles can be aligned with the separation device.

32. (canceled)

33. The system of claim 30, wherein the system comprises an inventory indicator that is a viewing window in the housing.

34-37. (canceled)

38. The system of claim 22, wherein the protrusion is angled with respect to the face.

39-40. (canceled)

41. The system of claim 22, wherein the protrusion is angled upward from a bottom plane of the dispenser housing.

42. The system of claim 41, further comprising a container implemented such that a rectangular prism has two end faces with a first area and four side faces with a second area, a perforated portion includes a portion of one of the four side face, the container holding the assembly of packaged articles.
